(19) United States
(12) Patent Application Publication
(10) Pub. No.: US 2025/0073240 A1
(43) Pub. Date: Mar. 6, 2025

(54) NEW TREATMENT

(71) Applicant: Verona Pharma PLC, Cardiff (GB)

(72) Inventors: Tara Renae Rheault, Cary, NC (US); Kathleen Rickard, Durham, NC (US)

(21) Appl. No.: 18/825,447

(22) Filed: Sep. 5, 2024

Related U.S. Application Data
(60) Provisional application No. 63/580,728, filed on Sep. 6, 2023.

(51) Int. Cl. A61K 31/519; A61K 9/00; A61K 45/06; A61K 47/02; A61K 47/26; A61P 11/00

(52) U.S. Cl. CPC A61K 31/519; A61K 9/0078; A61K 45/06; A61K 47/02; A61K 47/26; A61P 11/00

(57) ABSTRACT

The present disclosure relates to a compound for use in treating chronic obstructive pulmonary disease (COPD) in a patient, which compound is ensifentrine or a pharmaceutically acceptable salt thereof, wherein the patient has a blood eosinophil count of <300 cells/μL, <150 cells/μL, or <100 cells/μL.

### CROSS-REFERENCE

This application claims the benefit of U.S. Provisional Application No. 63/580,728, filed Sep. 6, 2023, which is incorporated herein by reference in its entirety.

### FIELD

The present invention relates to the treatment of chronic obstructive pulmonary disease (COPD).

### BACKGROUND

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

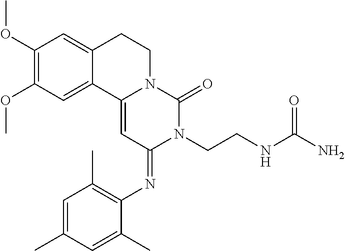 [SMILES: COc1cc2c(cc1OC)-c1c/c(=N\c3c(C)cc(C)cc3C)n(CCNC(N)=O)c(=O)n1CC2]

COPD is a progressive, long-term condition affecting a large number of people worldwide. Ongoing symptoms include breathlessness and cough. However, many COPD patients also suffer from occasional, temporary worsening of their symptoms (known as a COPD exacerbation). A COPD exacerbation typically involves an increase in breathlessness (dyspnea), the presence of excessive mucus (increased sputum volume), change in mucus colour (sputum purulence) and/or an increase in coughing.

Some patients with COPD are more difficult to treat than others. This includes patients having a low blood eosinophil count of <300 cells/μL. This may also include patients having blood eosinophil counts of, for example, <150 cells/μL or <100 cells/μL. Suissa (“Single-inhaler triple versus dual bronchodilator therapy for GOLD E and other exacerbating patients with COPD: Real-world comparative effectiveness and safety”, European Respiratory Journal 2023) explains that when seeking to combine anti-inflammatory and bronchodilatory activity in the treatment of COPD in this patient group using a triple combination of a long-acting muscarinic antagonist (LAMA), long-acting beta2-agonist (LABA) and inhaled corticosteroid (ICS), satisfactory results relating to both efficacy and safety were not obtained. On that basis, Suissa specifically recommends against using triple LAMA-LABA-ICS therapy in patients with a blood eosinophil count of less than 300 cells/μL.

There is a need to identify a safe and effective treatment for COPD in patients with a blood eosinophil count of <300 cells/μL (e.g., <150 cells/μL or <100 cells/μL).

### SUMMARY

Ensifentrine may be safe and effective in the treatment of COPD in patients with a blood eosinophil count of less than 300 cells/μL (e.g., <150 cells/μL or <100 cells/μL), in contrast to inhaled corticosteroid-containing therapies. Inhalable corticosteroids are often used in COPD to prevent exacerbations, despite an increased risk of pneumonia, including increased risk of hospitalized pneumonia. In some instances, this risk is even greater in the nearly half of COPD patients with lower levels of blood eosinophils, such as less than 150 or less than 100 cells/μL. A significant reduction in COPD exacerbations may be observed in this patient group using the combined anti-inflammatory and bronchodilator ensifentrine. Ensifentrine accordingly may provide a suitable treatment for COPD in patients with a blood eosinophil count of less than 300 cells/μL. Ensifentrine accordingly may also be a suitable treatment for COPD in patients with a blood eosinophil count of less than 150 cells/μL or 100 cells/μL.

In some embodiments, provided herein is a compound for use in a method of treating chronic obstructive pulmonary disease (COPD) in a patient, which compound is ensifentrine or a pharmaceutically acceptable salt thereof, wherein the patient has a blood eosinophil count of <300 cells/μL. In some embodiments, the compound may be for use in a method of treating COPD in a patient with a blood eosinophil count of <150 cells/μL. In some embodiments, the compound may be for use in a method of treating COPD in a patient with a blood eosinophil count of <100 cells/μL.

In some embodiments, provided herein is a method of treating chronic obstructive pulmonary disease (COPD) in a patient, the method comprising administering a therapeutically effective amount of a compound to the patient, which compound is ensifentrine or a pharmaceutically acceptable salt thereof, wherein the patient has a blood eosinophil count of <300 cells/μL, for example a blood eosinophil count of <150 cells/μL, or for example a blood eosinophil count of <100 cells/μL.

Also provided herein in some embodiments is a use of a compound in the manufacture of a medicament for use in a method of treating chronic obstructive pulmonary disease (COPD) in a patient, which compound is ensifentrine or a pharmaceutically acceptable salt thereof, wherein the patient has a blood eosinophil count of <300 cells/μL, for example a blood eosinophil count of <150 cells/μL.

In some embodiments, provided herein is a method of treating chronic obstructive pulmonary disease (COPD) in a patient comprising administering to the patient ensifentrine or a pharmaceutically acceptable salt thereof, wherein the patient has a blood eosinophil count of less than 300 cells/μL, for example a blood eosinophil count of <150 cells/μL, wherein the method reduces frequency of COPD exacerbations or severity of COPD exacerbations in the subject compared to frequency of COPD exacerbations or severity of COPD exacerbations in an untreated subject having COPD and not administered the composition. In some embodiments, the method decreases the frequency of COPD exacerbations or the severity of COPD exacerbations in the subject by at least 20% compared to the frequency of COPD exacerbations or the severity of COPD exacerbations in the untreated subject having COPD and not administered the composition. In some embodiments, the method decreases the frequency of COPD exacerbations or the severity of COPD exacerbations in the subject by at least 40% compared to the frequency of COPD exacerbations or the severity of COPD exacerbations in the untreated subject having COPD and not administered the composition.

In some embodiments, the method increases time to first COPD exacerbation in the subject compared to time to first COPD exacerbation in the untreated subject having COPD and not administered the composition.

In some embodiments, the subject has a blood eosinophil count of from about 170 cells/μL to about 260 cells/μL. In some embodiments, the subject has a blood eosinophil count of less than 150 cells/μL. In some embodiments, the subject has a blood eosinophil count of less than 100 cells/μL.

In some embodiments, the blood eosinophil count is determined from a sample of blood obtained from the subject using a Coulter counter.

In some embodiments, the subject is susceptible to COPD exacerbations. In some embodiments, the subject has suffered one or more COPD exacerbations in a year preceding the administering to the subject.

In some embodiments, the subject is susceptible to pneumonia.

In some embodiments, the subject is not on an inhaled corticosteroid regimen at a time of the administering to the subject.

In some embodiments, the composition is administered to the subject via inhalation.

In some embodiments, the administering to the subject is once, twice or three times per day. In some embodiments, the administering to the subject is twice a day.

In some embodiments, the administering to the subject comprises a total daily dose of the ensifentrine or the pharmaceutically acceptable salt thereof of from 0.5 to 10 mg. In some embodiments, the administering to the subject comprises a dose of about 3 mg of the ensifentrine or the pharmaceutically acceptable salt thereof to the subject twice a day.

In some embodiments, the administering to the subject comprises administering the ensifentrine or the pharmaceutically acceptable salt thereof to the subject at least once per day for at least 8 weeks, at least 16 weeks, or for at least 24 weeks.

In some embodiments, the method comprises administering an inhalable pharmaceutical composition comprising the ensifentrine or the pharmaceutically acceptable salt thereof in a diluent.

In some embodiments, the inhalable pharmaceutical composition comprises:
(a) particles comprising the ensifentrine or pharmaceutically acceptable salt thereof at a concentration of 1.2 mg/mL;
(b) polysorbate 20 (Tween 20) at a concentration of 0.5 mg/mL;
(c) sorbitan monolaurate (Span 20) at a concentration of 0.05 mg/mL;
(d) sodium dihydrogen phosphate dihydrate at a concentration of 0.744 mg/mL;
(e) disodium hydrogen phosphate dihydrate at a concentration of 0.853 mg/mL; and
(f) sodium chloride at a concentration of 8.6 mg/mL.

In some embodiments, the ensifentrine or the pharmaceutically acceptable salt thereof is used in combination with a muscarinic receptor antagonist, a beta-adrenergic receptor agonist or an inhaled corticosteroid.

In some embodiments, the ensifentrine or the pharmaceutically acceptable salt thereof is used in absence of a muscarinic receptor antagonist, a beta-adrenergic receptor agonist or an inhaled corticosteroid.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIG. 1 shows rate ratio of subjects with varying blood eosinophil counts.

FIG. 2 shows hazard ratio of time to first moderate/severe exacerbation of subjects with varying blood eosinophil counts.

### DETAILED DESCRIPTION

### Certain Definitions

As used herein and in the appended claims, the singular forms “a,” “and,” and “the” include plural referents unless the context clearly dictates otherwise. Thus, for example, reference to “an agent” includes a plurality of such agents. When ranges are used herein for physical properties, such as molecular weight, or chemical properties, such as chemical formulae, all combinations and subcombinations of ranges and specific embodiments therein are intended to be included. The term “comprising” (and related terms such as “comprise” or “comprises” or “having” or “including”) is not intended to exclude. An embodiment of any composition of matter, composition, method, or process, or the like, described herein, may “consist of” or “consist essentially of” the described features. As used herein, the term “about” may represent a variation of ±10% of the stated value.

As used herein, unless stated otherwise, “%” or “percent” as used herein means percent by weight (e.g., w/w %), percent by volume (e.g., v/v %). Preferably, “%” or “percent” as used herein means percent by weight (e.g., w/w %).

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

### Methods of Treating COPD & COPD Exacerbations

The compound may be used in a method of treating COPD.

The compound may decrease the frequency and/or severity of chronic obstructive pulmonary disease (COPD) exacerbations in a patient suffering from COPD. The compound may decrease the frequency of COPD exacerbations. The compound may decrease the severity of COPD exacerbations.

For instance, the patient may suffer two or fewer (for instance one or zero) COPD exacerbations per year while being treated with the compound, for instance as a maintenance therapy. In some embodiments, the patient may suffer five or fewer COPD exacerbations per year while being treated with the compound. In some embodiments, the patient may suffer three or fewer COPD exacerbations per year while being treated with the compound, for instance as a maintenance therapy. In some embodiments, the patient may suffer one COPD exacerbation per year while being treated with the compound, for instance as a maintenance therapy. In some embodiments, the patient may suffer zero COPD exacerbations per year while being treated with the compound, for instance as a maintenance therapy.

In some embodiments, COPD exacerbations are reduced by at least 20% while being treated with the compound, for instance as a maintenance therapy. In some embodiments, COPD exacerbations are reduced by at least 30% (e.g., at least 32%, at least 34%, at least 36%, at least 38%, at least 40%, at least 42%, or at least 44%) while being treated with the compound, for instance as a maintenance therapy.

In some embodiments, moderate COPD exacerbations are reduced by at least 20% while being treated with the compound, for instance as a maintenance therapy. In some embodiments, moderate COPD exacerbations are reduced by at least 30% (e.g., at least 32%, at least 34%, at least 36%, at least 38%, at least 40%, at least 42%, or at least 44%) while being treated with the compound, for instance as a maintenance therapy.

In some embodiments, severe COPD exacerbations are reduced by at least 20% while being treated with the compound, for instance as a maintenance therapy. In some embodiments, severe COPD exacerbations are reduced by at least 30% (e.g., at least 32%, at least 34%, at least 36%, at least 38%, at least 40%, at least 42%, or at least 44%) while being treated with the compound, for instance as a maintenance therapy.

As used herein, rate reduction may be determined via 1—rate ratio, such as a rate ratio exemplified elsewhere herein.

In some embodiments, such as described in Table 5, following treatment with a pharmaceutical composition described herein, a rate of COPD exacerbations may be decreased by at least 40% (e.g., at least 42%, 43%, 44%, 45%, 46%, or 47%) in subjects with a blood eosinophil count of <300 cells/μL. In some embodiments, a rate of COPD exacerbations may be decreased by at least 45% in subjects with a blood eosinophil count of <300 cells/μL. In some embodiments, a rate of COPD exacerbations may be decreased by from about 30% to about 60% (e.g., from about 30% to about 50%, from about 35% to about 45%, from about 40% to about 50%) in subjects with a blood eosinophil count of <300 cells/μL. In some embodiments, a rate of COPD exacerbations may be decreased by about 40%, 41%, 42%, 43%, 44%, 45%, 46%, 47%, 48%, 49%, 50%, 51%, 52%, 53%, 54%, or about 55% in subjects with a blood eosinophil count of <300 cells/μL.

In some embodiments, such as described in Table 3, following treatment with a pharmaceutical composition described herein, a rate of COPD exacerbations may be decreased by at least 35% (e.g., at least 36%, 37%, 38%, 39%, 40%, 41%, or 42%) in subjects with a blood eosinophil count of <150 cells/μL. In some embodiments, a rate of COPD exacerbations may be decreased by at least 40% in subjects with a blood eosinophil count of <150 cells/μL. In some embodiments, a rate of COPD exacerbations may be decreased by from about 30% to about 60% (e.g., from about 30% to about 50%, from about 35% to about 45%, from about 40% to about 50%) in subjects with a blood eosinophil count of <150 cells/μL. In some embodiments, a rate of COPD exacerbations may be decreased by about 40%, 41%, 42%, 43%, 44%, 45%, 46%, 47%, 48%, 49%, 50%, 51%, 52%, 53%, 54%, or about 55% in subjects with a blood eosinophil count of <150 cells/μL.

In some embodiments, such as described in Table 7, following treatment with a pharmaceutical composition described herein, a rate of COPD exacerbations may be decreased by at least 40% (e.g., at least 41%, 42%, 43%, 44%, 45%, 46%, 47%, or 48%) in subjects with a blood eosinophil count of <100 cells/μL. In some embodiments, a rate of COPD exacerbations may be decreased by at least 45% in subjects with a blood eosinophil count of <100 cells/μL. In some embodiments, a rate of COPD exacerbations may be decreased by from about 30% to about 60% (e.g., from about 30% to about 50%, from about 35% to about 45%, from about 40% to about 50%) in subjects with a blood eosinophil count of <100 cells/μL. In some embodiments, a rate of COPD exacerbations may be decreased by about 40%, 41%, 42%, 43%, 44%, 45%, 46%, 47%, 48%, 49%, 50%, 51%, 52%, 53%, 54%, or about 55% in subjects with a blood eosinophil count of <100 cells/μL. In some embodiments, risk of (e.g., moderate or severe) COPD exacerbations are decreased by at least 30% vs. placebo after treatment with the compound, such as in subjects with a blood eosinophil count of <300 cells/μL. In some embodiments, risk of (e.g., moderate or severe) COPD exacerbations are decreased by at least 40% vs. placebo after treatment with the compound, such as in subjects with a blood eosinophil count of <300 cells/μL. In some embodiments, risk of (e.g., moderate or severe) COPD exacerbations are decreased by at least 50% vs. placebo after treatment with the compound, such as in subjects with a blood eosinophil count of <300 cells/μL. In some embodiments, risk of (e.g., moderate or severe) COPD exacerbations are decreased by about 30% to about 50% vs. placebo after treatment with the compound, such as in subjects with a blood eosinophil count of <300 cells/μL. In some embodiments, risk of (e.g., moderate or severe) COPD exacerbations are decreased by about 50% vs. placebo after treatment with the compound, such as in subjects with a blood eosinophil count of <300 cells/μL (e.g., such as described in the Examples elsewhere herein).

In some embodiments, risk of (e.g., moderate or severe) COPD exacerbations are decreased by at least 30% vs. placebo after treatment with the compound, such as in subjects with a blood eosinophil count of <150 cells/μL. In some embodiments, risk of (e.g., moderate or severe) COPD exacerbations are decreased by at least 40% vs. placebo after treatment with the compound, such as in subjects with a blood eosinophil count of <150 cells/μL. In some embodiments, risk of (e.g., moderate or severe) COPD exacerbations are decreased by at least 50% vs. placebo after treatment with the compound, such as in subjects with a blood eosinophil count of <150 cells/μL. In some embodiments, risk of (e.g., moderate or severe) COPD exacerbations are decreased by about 30% to about 50% vs. placebo after treatment with the compound, such as in subjects with a blood eosinophil count of <150 cells/μL. In some embodiments, risk of (e.g., moderate or severe) COPD exacerbations are decreased by about 40% vs. placebo after treatment with the compound, such as in subjects with a blood eosinophil count of <150 cells/μL (e.g., such as described in the Examples elsewhere herein).

In some embodiments, risk of (e.g., moderate or severe) COPD exacerbations are decreased by at least 30% vs. placebo after treatment with the compound, such as in subjects with a blood eosinophil count of <100 cells/μL. In some embodiments, risk of (e.g., moderate or severe) COPD exacerbations are decreased by at least 40% vs. placebo after treatment with the compound, such as in subjects with a blood eosinophil count of <100 cells/μL. In some embodiments, risk of (e.g., moderate or severe) COPD exacerbations are decreased by at least 50% vs. placebo after treatment with the compound, such as in subjects with a blood eosinophil count of <100 cells/μL. In some embodiments, risk of (e.g., moderate or severe) COPD exacerbations are decreased by about 30% to about 50% vs. placebo after treatment with the compound, such as in subjects with a blood eosinophil count of <100 cells/μL. In some embodiments, risk of (e.g., moderate or severe) COPD exacerbations are decreased by about 40% vs. placebo after treatment with the compound, such as in subjects with a blood eosinophil count of <100 cells/μL (e.g., such as described in the Examples elsewhere herein). In some instances, risk of (e.g., moderate or severe) COPD exacerbations are relative to placebo.

The number of COPD exacerbations experienced by the patient per year during treatment with the compound may be one to three fewer than the number of COPD exacerbations experienced by the patient per year prior to treatment with the compound (for instance per year in the two years prior to treatment with the compound). In some embodiments, the number of COPD exacerbations experienced by the patient per year during treatment with the compound may be at least one (e.g., at least two, at least three, at least four, or at least five) fewer than the number of COPD exacerbations experienced by the patient per year prior to treatment with the compound (for instance per year in the two years prior to treatment with the compound).

In some embodiments, the compound may increase the time to a first COPD exacerbation in the patient. Accordingly, the patient may not have yet experienced a COPD exacerbation and the compound may increase the time until the patient experiences a first COPD exacerbation (i.e. the first COPD exacerbation is delayed).

The compound may accordingly reduce the risk of COPD exacerbations in a COPD patient.

In some embodiments, the COPD exacerbations comprise one or more of dyspnea (breathlessness), increased coughing, increased sputum volume, sputum purulence, wheezing, sore throat, a cold, and fever. Sputum purulence is a change in the colour of spontaneously expectorated samples from uncoloured to yellow-green. The COPD exacerbation may last for at least one day or at least two days. In some instances, the COPD exacerbation lasts for at least one day. In other instances, the COPD exacerbation lasts for at least two days.

A COPD exacerbation may comprise (A) worsening of two or more of the following major symptoms for at least two consecutive days: dyspnea, sputum volume and sputum purulence or (B) worsening of any one major symptom together with any one of the following minor symptoms for at least two consecutive days: sore throat, colds (nasal discharge and/or nasal congestion), fever (oral temperature >37.5° C.) without other cause and increased cough. In some embodiments, a COPD exacerbation comprises worsening of two or more of dyspnea, sputum volume and sputum purulence. In some embodiments, a COPD exacerbation comprises worsening of any one major symptom together with any one of the following minor symptoms for at least two consecutive days: sore throat, colds (nasal discharge and/or nasal congestion), fever (oral temperature >37.5° C.) without other cause and increased cough. For instance, a COPD exacerbation may comprise worsening of two or more of the major symptoms (dyspnea, sputum volume and sputum purulence) for at least two consecutive days.

A COPD exacerbation may be a moderate COPD exacerbation or a severe COPD exacerbation. A moderate exacerbation may be defined as worsening symptoms of COPD (as defined above) requiring a minimum of three days of treatment with oral/systemic corticosteroids and/or antibiotics. A severe exacerbation may be defined as worsening symptoms of COPD (as defined above) requiring in-patient hospitalization.

In some embodiments, the compound may reduce the severity of COPD exacerbations in a patient, and accordingly the compound may be for use in preventing severe COPD exacerbations in a patient. For instance, the patient may experience no severe COPD exacerbations in the year following first administration of the compound. The compound may be for preventing moderate COPD exacerbations in a patient.

In some embodiments, the compound may lengthen the time to a COPD exacerbation. In some embodiments, the compound may lengthen the time to a COPD exacerbation by at least one month. For instance, the compound may increase the time to an exacerbation by two or more months. In some embodiments, the compound may lengthen the time to a COPD exacerbation by at least three, four, five, or six months.

In some embodiments, the patient is susceptible to COPD exacerbations. In some embodiments, the patient susceptible to COPD exacerbations is a patient suffering from one or more co-morbidities (other than COPD). In some embodiments, said patient is suffering from one or more disease or condition selected from asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease or tuberculosis, optionally wherein the asthma is allergic asthma, steroid resistant asthma, severe asthma or paediatric asthma. The patient susceptible to COPD exacerbation may have a chronic bronchitis etiology, may have impaired lung function (such as 30 to 70% predicted FEV₁) or may have COPD symptoms despite use of long-acting muscarinic receptor antagonist (LAMA) or a long-acting beta-adrenergic receptor agonist (LABA) therapy.

In some embodiments, the compound may be for use in treating COPD in a patient suffering from asthma. In some embodiments, the compound may be for use in treating COPD in a patient having a blood eosinophil count of <300 cells/μL, for example a blood eosinophil count of <300 cells/μL. In some embodiments, the compound may be for use in treating COPD in a patient suffering from pulmonary hypertension. In some embodiments, the compound may be for use in treating COPD in a patient suffering from bronchiectasis.

For instance, the compound may be for use in treating COPD in a patient suffering from both COPD and asthma, and having a blood eosinophil count of <300 cells/μL. The compound may be for use in treating COPD a patient suffering from both COPD and pulmonary hypertension. The compound may be for use in treating COPD in a patient suffering from both COPD and bronchiectasis.

Other risk factors for COPD exacerbation may include: high serum immunoglobulin (Ig), previous tuberculosis, severe airflow obstruction, chest infection and one or more hospital admission for COPD exacerbations in the previous year.

In some instances, a patient experiencing COPD exacerbations may have high serum immunoglobulin (Ig). In some instances, a patient experiencing COPD exacerbations may have had previous tuberculosis. In some instances, a patient experiencing COPD exacerbations may have severe airflow obstruction. In some instances, a patient experiencing COPD exacerbations may have had a chest infection and one or more hospital admission for COPD exacerbations in the previous year.

In some cases, the patient has suffered one or more COPD exacerbations in the year preceding first administration of the compound. For instance, the patient may have suffered two or more COPD exacerbations in the year preceding first administration of the compound. In some embodiments, the patient may for instance have suffered from at least one severe COPD exacerbation (i.e. requiring hospital treatment) in the year preceding first administration of the compound. In some embodiments, the patient may have suffered one or more COPD exacerbations in the six months preceding first administration of the compound, or one or more COPD exacerbations in the one month preceding first administration of the compound.

Administration of the compound may not be associated with an increased risk of pneumonia. In some embodiments, after administration of the compound to the patient, the patient is not susceptible/has decrease susceptibility to pneumonia. There are a number of factors which may cause susceptibility to pneumonia. For instance, the patient may be aged 65 years or older, 75 years or older or 85 years or older. The patient may have a BMI of at least 30, at least 35 or at least 40.

In some embodiments, the patient may be suffering from a disease or condition associated with an increased risk of pneumonia. For instance, the patient susceptible to pneumonia may be suffering from one or more disease or condition selected from diabetes, dementia, Parkinson's disease, cystic fibrosis, cancer, multiple sclerosis and osteoporosis. In some embodiments, the patient susceptible to pneumonia may have suffered a stroke or transient ischaemic attack.

### Blood Eosinophil

In some embodiments, a patient described herein that is administered the compound has a blood eosinophil count of <300 cells/μL. In some embodiments, the patient has a blood eosinophil count of <200 cells/μL. In some embodiments, the patient has a blood eosinophil count of <150 cells/μL. For instance, the patient may have a blood eosinophil count of from 10 to 300 cells/μL, or from 30 to 280 cells/μL. In some embodiments, the patient may have a blood eosinophil count of from about 1 to about 300 cells/μL, about 10 to about 300 cells/μL, about 20 to about 290 cells/μL, about 30 to about 280 cells/μL, about 50 to about 260 cells/μL, about 10 to about 250 cells/μL, about 25 to about 250 cells/μL, about 25 to about 300 cells/μL, about 50 to about 300 cells/μL, about 100 to about 300 cells/μL, about 75 to about 250 cells/μL, about 100 to about 250 cells/μL, about 100 to about cells/μL, about 100 to about 300 cells/μL, or about 200 to about 300 cells/μL. The patient may have a blood eosinophil count of <275 cells/μL. In some embodiments, the patient has a blood eosinophil count of <290 cells/μL (e.g., less than 280 cells/μL, 270 cells/μL, 260 cells/μL, 250 cells/μL, 240 cells/μL, 230 cells/μL, 220 cells/μL, 210 cells/μL, 200 cells/μL, 180 cells/μL, 160 cells/μL, or 150 cells/μL). The patient may have a blood eosinophil count of >160 cells/μL, for instance from 160 to 300 cells/μL. In some embodiments, the patient has a blood eosinophil count of >170 cells/μL (e.g., greater than 180 cells/μL, 190 cells/μL, 200 cells/μL, 210 cells/μL, 220, cells/μL, 240 cells/μL, 250 cells/μL, 260 cells/μL, 280 cells/μL, or greater than 290 cells/μL). In some embodiments, the patient may have a blood eosinophil count of from 100 to 280 cells/μL, for instance from 170 to 270 cells/μL.

In some embodiments, the blood eosinophil count is an average blood eosinophil count.

In some embodiments, the patient may have a blood eosinophil count of <200 cells/μL. In some embodiments, the patient may have a blood eosinophil count of <150 cells/μL, for instance from 20 to 150 cells/μL.

### Blood Eosinophil Determination

The blood eosinophil count of the patient may be as determined by any suitable means. In some embodiments, a complete blood count and white blood cell differential may be determined. The blood eosinophil count may be determined based on a sample of the patient's blood. In some embodiments, the blood eosinophil count of the patient is as determined from a sample of the patient's blood using the Coulter principle. The Coulter principle is a well-known means for counting and determining the size of blood cells suspended in an electrolyte. The Coulter principle states that particles pulled through an orifice, concurrent with an electric current, produce a change in impedance proportional to the volume of the particle traversing the orifice. In some instances, the blood eosinophil count of the patient is as determined using a Coulter counter, for instance a Beckman Coulter DxH 800. The blood eosinophil count of the patient may be as determined by (a) taking a 2 mL blood sample from the patient and mixing it with 2 mL of a solution comprising EDTA and (b) measuring the blood eosinophil count as part of the white blood cell (WBC) differential using a Coulter counter at ambient temperature (25° C.). In some embodiments, the Coulter counter may be a Beckman Coulter DxH 800.

The blood eosinophil count may alternatively be as determined by microscope analysis of a smear of the patient's blood on a slide.

In some embodiments, a patient's blood eosinophil count (e.g., as described elsewhere herein) may be as measured within one month prior to first administration of the compound. In some embodiments, a patient's blood eosinophil count (e.g., as described elsewhere herein) may be as measured within one week prior to first administration of the compound. In some embodiments, a patient's blood eosinophil count (e.g., as described elsewhere herein) may be as measured within two weeks prior to first administration of the compound.

In some embodiments, a patient may have an average blood eosinophil count as described elsewhere herein over a period of one week prior to first administration of the compound. In some embodiments, a patient may have an average blood eosinophil count as described elsewhere herein over a period of two weeks prior to first administration of the compound. In some embodiments, a patient may have an average blood eosinophil count as described elsewhere herein over a period of three weeks prior to first administration of the compound. The average blood eosinophil count may be as determined by taking at least two measurements of blood eosinophil count using a Coulter counter within one week prior to first administration, which at least two measurements are taken at least 24 hours apart. In some embodiments, the patient may have a blood eosinophil count of <300 cells/μL as measured within one month prior to first administration of the compound, preferably within one week prior to first administration of the compound. In some embodiments, the patient may have an average blood eosinophil count of <300 cells/μL over a period of one week prior to first administration of the compound. In some embodiments, the average blood eosinophil count may be as determined by taking at least two measurements of blood eosinophil count using a Coulter counter within one week prior to first administration, which at least two measurements are taken at least 24 hours apart.

### Blood Eosinophil Patient Selection

The methods disclosed herein may further comprise determining the blood eosinophil count of the patient. In some embodiments, the method comprises determining the blood eosinophil count of the patient using a Coulter counter. In some embodiments, the method comprises determining the blood eosinophil count of the patient prior to first administration of the compound. The method may further comprise selecting the patient for treatment with the compound based on the blood eosinophil count of the patient.

In some embodiments, the method comprises selecting the patient for treatment with the compound if the blood eosinophil count of the patient is determined to be within a pre-determined range. In some instances, the predetermined range is <300 cells/μL. In some instances, the predetermined range is <150 cells/μL. In some embodiments, the predetermined range is <100 cells/μL. In some instances, the predetermined range is from about 1 to about 300 cells/μL, about 10 to about 300 cells/μL, about 20 to about 290 cells/μL, about 30 to about 280 cells/μL, about 50 to about 260 cells/μL, about 10 to about 250 cells/μL, about 25 to about 250 cells/μL, about 25 to about 300 cells/μL, about 50 to about 300 cells/μL, about 100 to about 300 cells/μL, about 75 to about 250 cells/μL, about 100 to about 250 cells/μL, about 100 to about cells/μL, about 100 to about 300 cells/μL, or about 200 to about 300 cells/μL. In some instances, the predetermined range is <290 cells/μL (e.g., less than 280 cells/μL, 270 cells/μL, 260 cells/μL, 250 cells/μL, 240 cells/μL, 230 cells/μL, 220 cells/μL, 210 cells/μL, 200 cells/μL, 180 cells/μL, 160 cells/μL, or 150 cells/μL). In some instances, the predetermined range is >170 cells/μL (e.g., greater than 180 cells/μL, 190 cells/μL, 200 cells/μL, 210 cells/μL, 220, cells/μL, 240 cells/μL, 250 cells/μL, 260 cells/μL, 280 cells/μL, or greater than 290 cells/μL).

The method may comprise (i) determining the blood eosinophil count of the patient using a Coulter counter and (ii) administering the compound to the patient for the treatment of COPD if the blood eosinophil count of the patient is determined to be <300 cells/μL (for instance <150 cells/μL).

### Patients

In some embodiments, a subject disclosed herein is a patient. In some embodiments, the subject is a mammal. In some embodiments, the subject is a human. In some embodiments, the subject is a human male. In some embodiments, the subject is a human female. In some embodiments, the subject is greater than or equal to 65 years of age. In some embodiments, the subject is less than 65 years of age. In some embodiments, the subject is greater than 18 years of age. In some embodiments, the human subject has a BMI of at least 30.

In some embodiments, the subject has a blood eosinophil count of less than 300 cells/μL. In some embodiments, the subject has a blood eosinophil count of less than 200 cells/μL. In some embodiments, the subject has a blood eosinophil count of less than 150 cells/μL. In some embodiments, the subject has a blood eosinophil count of less than 100 cells/μL.

Ensifentrine has been found to be a safe and effective treatment for a subgroup of COPD patients for which inhaled corticosteroids are not suitable. As such, the patient may not be receiving an inhaled corticosteroid at the time of treatment with the compound. In some embodiments, the patient has not received an inhaled corticosteroid within 1 week of first being administered the compound. In some embodiments, the patient has not received an inhaled corticosteroid within 1 month, within 6 months or within 1 year of first being administered the compound. The inhaled corticosteroid may comprise one or more inhaled corticosteroids selected from beclomethasone, budesonide, ciclesonide, flunisolide, fluticasone, mometasone, triamcinolone and salts or esters thereof. Thus, the patient may not have received beclomethasone, budesonide, ciclesonide, flunisolide, fluticasone, mometasone, triamcinolone or a salt or ester thereof within 6 months from first treatment with the compound.

In some embodiments, the patient may be male. In some embodiments, the patient may be female. In some embodiments, the patient may have an age of greater than or equal to 65 years. In some embodiments, the patient may have an age of less than 65 years. In some embodiments, the patient may be taking a background medication selected from one or more of a long-acting muscarinic antagonist (LAMA) and a long-acting beta-agonist (LABA).

### Compound Administration

In some embodiments, the compound is ensifentrine or a pharmaceutically acceptable salt thereof. Pharmaceutically acceptable salts are well known to the skilled person. In some embodiments, the compound is ensifentrine (i.e., ensifentrine free base).

In some embodiments, the method comprises administering the compound to the patient by inhalation.

In some embodiments, a pharmaceutical composition comprising the compound and one or more pharmaceutically acceptable excipients or diluents is administered to the patient by inhalation, for instance by nebuliser, pressurised metered dose inhaler (pMDI) or dry powder inhaler (DPI). In some embodiments, the pharmaceutical composition is administered to the patient by inhalation by nebuliser. In some embodiments, the pharmaceutical composition is administered to the patient by inhalation by pressurised metered dose inhaler (pMDI). In some embodiments, the pharmaceutical composition is administered to the patient by inhalation by dry powder inhaler (DPI).

In some embodiments, the method comprises administering the compound to the patient by inhalation from a nebuliser. Nebulisers aerosolise a liquid pharmaceutical composition into an aerosol that is inhaled into a patient's respiratory tract. Examples of nebulisers include a soft mist nebuliser, a vibrating mesh nebuliser, a jet nebuliser and an ultrasonic wave nebuliser. Suitable nebuliser devices include the Philips I-neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The compound may be inhaled via the nebuliser for from 1 to 15 minutes. The compound may be inhaled via the nebulizer for from about 1 to about 15 minutes. In some embodiments, the compound may be inhaled via the nebulizer for at least 1 minute. In some embodiments, the compound may be inhaled via the nebulizer for about 1 minute to about 12 minutes, about 1 minute to about 8 minutes, about 1 minute to about 4 minutes, about 3 minutes to about 15 minutes, about 4 minutes to about 10 minutes, or about 6 minutes to about 12 minutes. In some embodiments, the compound may be inhaled via the nebulizer for from 5 to 7 minutes. In some embodiments, the compound may be inhaled for 5 minutes. In some embodiments, the compound may be inhaled via the nebulizer for 6 minutes. In some embodiments, the compound may be inhaled via the nebulizer for 7 minutes.

In some embodiments, the method comprises administering the compound to the patient once, twice or three times per day, for instance twice or three times per day. The compound may be administered to the patient by inhalation once, twice or three times a day. In some embodiments, the method comprises administering the compound to the patient by inhalation twice a day. The method may comprise administering a first dose of the compound in the morning (for instance within 3 hours following waking) and a second dose of the compound in the evening (for instance within 3 hours before bed). In some embodiments, the morning and evening doses are administered from 10 to 14 hours apart, for instance about 12 hours apart.

The compound may be used in any suitable therapeutically effective amount. In some embodiments, the daily dose of the compound is from 0.1 to 20 mg. In some embodiments, the method comprises administering a total daily dose of the compound of from 0.5 to 10 mg. In some embodiments, the total daily dose of the compound (e.g., ensifentrine free base) is from 5 to 7 mg, for instance about 6 mg per day. The total daily dose of the compound may be 6.0 mg.

In some embodiments, the compound is administered twice a day in two separate doses which are the same or similar. For instance, the method may comprise administering the compound to the patient twice a day in a first dose of from 1 to 5 mg and a second dose of from 1 to 5 mg. In some embodiments, the method may comprise administering the compound to the patient twice a day in a first dose of from 2 to 4 mg and a second dose of from 2 to 4 mg.

In some embodiments, the method comprises administering two doses of about 3 mg ensifentrine free base to the patient per day by inhalation. The method may comprise administering a dose of about 3 mg of the compound to the patient twice a day (3 mg BID) by inhalation. The method may comprise administering by nebuliser a dose of about 3 mg the compound to the patient twice a day. Each dose may be 3.0 mg free base ensifentrine administered by nebulizer.

The compound may be used as a maintenance therapy. In some embodiments, the method comprises administering the compound to the patient at least once per day for at least 8 weeks. The compound may be administered to the patient at least once per day for at least 16 weeks, preferably for at least 24 weeks. The compound may be administered daily to the patient for at least 1 year. The method may comprise administering the compound to the patient at least once every 24 hours, preferably at least twice every 24 hours, for at least 8 weeks, preferably for at least 16 weeks, more preferably for at least 24 weeks.

### Ensifentrine Pharmaceutical Compositions

In some instances, the compound (e.g., ensifentrine) provided herein is comprised in a pharmaceutical composition described herein.

The compound may be administered as a suspension formulation, i.e., a suspension of particles comprising the compound in a diluent. The compound may alternatively be delivered as a dry powder, for instance a dry powder comprising particles comprising the compound and particles of a carrier such as lactose.

A method disclosed herein may comprise administering an (e.g., inhalable) pharmaceutical composition comprising a suspension of particles of the compound in a diluent. The particles comprising the compound may have a particle size distribution with a Dv50 of from 0.5 μm to 5.0 μm. In some embodiments, the particles have a Dv50 of from 1.0 μm to 2.0 μm. In some embodiments, the particles comprise a Dv90 of from about 1.8 μm to about 2 μm. In some embodiments, the particles comprise a Dv90 of from about 2.5 μm to about 6.0 μm. In some embodiments, the particles comprise a Dv90 of from about 2.8 μm to about 4.2 μm. In some embodiments, the particles comprise a Dv10 of from about 0.2 μm to about 1.0 μm. In some embodiments, the particles comprise a Dv10 of from about 0.4 μm to about 0.6 μm. In some embodiments, the particles comprise a Dv10 of from about 0.4 μm to about 0.6 μm and a Dv90 of from about 2.8 μm to about 4.2 μm. In certain embodiments, the particles comprise a Dv90 that is not more than 4.5 μm. In some embodiments, the particles comprise a Dv90 of about 2, 2.1, 2.2, 2.3, 2.5, 2.5, or 2.6 μm. Particle sizes are described herein by reference to the Dv50 value, which is the median particle size for a volume distribution. Thus, half the volume of the particles have diameters of less than the Dv50 value and half the volume of the particles have diameters of greater than the Dv50 value. Similarly, in some embodiments, particle sizes are described herein by their Dv90 or Dv10 values. This is a well-known manner in which to describe particle size distributions.

The technique used to measure the Dv50, Dv90, and Dv10 values as stated herein may be laser diffraction. The particle size distribution of the particles comprising the compound may be as measured by laser diffraction using a wet powder dispersion system. For instance, the particle size distribution can be measured by laser diffraction using a Malvern Spraytec in conjunction with a wet dispersion cell. In some embodiments, the instrument parameters for the Malvern Spraytec are as follows:
particle—standard opaque particle,
refractive index Particle—1.50;
refractive index (imaginary)—0.50;
density of particle—1.00;
refractive index of dispersant—1.33;
controller unit—1000 RPM;
measurement type—timed;
initial sampling time—30 s;
obscuration—20%-30%;
dispersant—1% Polysorbate 20 in deionised water.

The particles comprising the compound typically comprise ensifentrine (i.e. ensifentrine free base). The particles may comprise at least 90 wt % ensifentrine free base relative to the total weight of the particles. In some embodiments, the particles comprise at least 95 wt % ensifentrine free base relative to the total weight of the particles. In some embodiments, the particles comprise at least 97 wt % ensifentrine free base relative to the total weight of the particles. In some instances, the particles consist of ensifentrine. The particles may comprise at least 99 wt % ensifentrine. In some embodiments, the particles comprise at least 99.5 wt % ensifentrine free base relative to the total weight of the particles. The particles may consist of ensifentrine.

### Crystalline Form

In some embodiments, the compound (e.g., ensifentrine) provided herein is in crystalline form. In some embodiments, at least 90% of the ensifentrine by weight is in the form of ensifentrine free base Form I. In some embodiments, at least 90% of the particles comprise ensifentrine free base Form I. In some embodiments, Ensifentrine free base Form I is a crystalline polymorph of ensifentrine (crystalline polymorph Form I) which has a powder X-ray diffraction pattern comprising characteristic peaks at 10.1° and 12.9°±0.1° 2θ. As used herein, values of °2θ may be measured using an X-ray wavelength of Cu Kα radiation (λ=1.5406 Å). In some embodiments, the powder x-ray diffraction pattern of Form I further comprises characteristic peaks at 15.3° and 17.6°±0.1° 2θ. In some embodiments, Form I of ensifentrine may have a powder X-ray diffraction pattern comprising at least 5 characteristic peaks selected from 6.4°, 10.1°, 12.6°, 12.9°, 13.6°, 14.2°, 14.7°, 15.3°, 15.4°, 15.8°, 17.0°, 17.6°, 18.9°, 20.9°, 22.4°, 22.8° and 28.7°±0.1° 2θ. In some embodiments, crystalline polymorph Form I has a differential scanning calorimetry trace showing a maximum at 248° C.

In some embodiments, the powder x-ray diffraction pattern of Form I of ensifentrine comprises at least 5 (e.g., at least 3, 4, 6, 7, or 8) characteristic peaks, in terms of 2θ, selected from about 6.4°, about 10.1°, about 12.6°, about 12.9°, about 13.6°, about 14.2°, about 14.7°, about 15.3°, about 15.4°, about 15.8°, about 17.0°, about 17.6°, about 18.9°, about 20.9°, about 22.4°, about 22.8°, and about 28.7°.

In some embodiments, the particles comprise at least 98% by weight of ensifentrine crystalline polymorph Form I. In some embodiments, the (e.g., ensifentrine) particles comprise at least 99% by weight of ensifentrine crystalline polymorph Form I.

In some embodiments, ensifentrine crystalline polymorph Form I has the following structural parameters obtained by single crystal analysis:

| Wavelength | 0.71073 Å | |
|---|---|---|
| Crystal System | Triclinic | |
| Space Group | P-1 | |
| Unit Cell Dimensions | a = 8.1246(4) Å | α = 91.583(4)° |
| b | b = 11.4573(5) Å | β = 90.299(4)° |
| c | c = 13.2398(6) Å | γ = 99.628(4)° |
| Volume | 1214.56(10) Å 3 | |
| C | 2. | |

In some instances, the crystalline polymorph Form I is the most thermodynamically stable polymorphic form of ensifentrine and is also expected to have the longest shelf life storage. It is expected that this advantage should extend further to the potential shelf life of any commercial ensifentrine drug product comprising the stable crystalline form I as an API. Furthermore, crystalline polymorph I is more amenable for the development of a reproducible uniform micronized dry solid powder of ensifentrine for certain pharmaceutical formulation applications, such as preparation of formulations described herein.

### Impurity Profile

It has been found that known processes for producing ensifentrine are associated with undesirable levels of impurities. For example, the process described in WO 00/58308 A1 in which a urea group is added using sodium cyanate and aqueous hydrochloric acid produces and ensifentrine substance containing a biuret impurity which could not be readily removed. WO 2018/020249 A1 discusses several possible reagents which may be used to add a urea group in the final step of the production of ensifentrine, but no detailed discussion of the conditions of the final ureation step is provided. It may be desirable to utilize synthetic processes for the production of ensifentrine which produce a drug substance with a favorable impurity profile without the need for extensive purification and recrystallization, and where the drug substance comprises low levels of a biuret impurity. In some instances, it has been found that it is possible to produce ensifentrine with a favorable impurity profile, and ensifentrine having low levels of a biuret impurity, by reaction comprising reacting an amine intermediate with 4-nitrophenyl chloroformate and ammonia in a solvent comprising dichloromethane.

In some embodiments, the (e.g., ensifentrine comprising) particles provided herein comprise 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU). In some embodiments, the structure of BMIQU is:

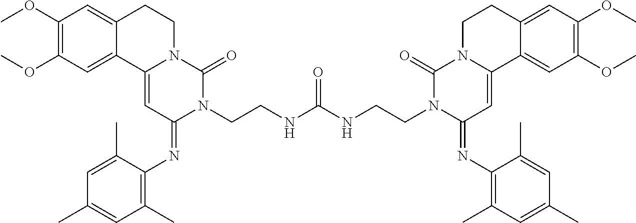

In some embodiments, the (e.g., ensifentrine comprising) particles provided herein comprise the BMIQU in an amount of from 0% to about 0.6% by weight. In some embodiments, the particles comprise BMIQU in an amount of at least about 0.005% by weight. In some embodiments, the particles comprise BMIQU in an amount of at least about 0.01% by weight. In some embodiments, the particles comprise BMIQU in an amount of at least about 0.1% by weight. In some embodiments, the particles comprise BMIQU in an amount of at most about 1% by weight. In some embodiments, the particles comprise BMIQU in an amount of at most about 0.6% by weight. In some embodiments, the particles comprise BMIQU in an amount of at most about 0.3% by weight. In some embodiments, the particles comprise BMIQU in an amount of from about 0.01% to about 0.3% by weight. In some embodiments, the particles comprise BMIQU in an amount of from about 0.3% to about 0.6% by weight. In some embodiments, the particles comprise BMIQU in an amount of from about 0.02% to about 0.06% by weight. In some embodiments, the weight percentage is relative to the total weight of ensifentrine (e.g., free base). In some embodiments, the weight percentage is relative to the total weight of the particles.

In some embodiments, the (e.g., ensifentrine comprising) particles provided herein comprise a biuret impurity of formula (A):

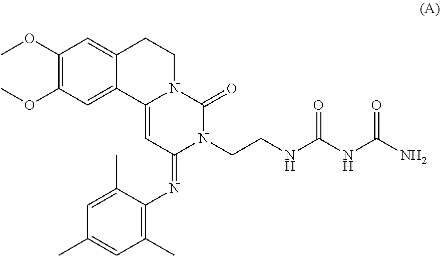 [SMILES: COc1cc2c(cc1OC)-c1c/c(=N\c3c(C)cc(C)cc3C)n(CCNC(=O)NC(N)=O)c(=O)n1CC2]

In some embodiments, the (e.g., ensifentrine comprising) particles comprise the biuret impurity of formula (A) in an amount of from 0% to about 0.5% by weight. In some embodiments, the particles comprise the biuret impurity of formula (A) in an amount of at least 0.01% by weight. In some embodiments, the particles comprise the biuret impurity of formula (A) in an amount of at least 0.05% by weight. In some embodiments, the particles comprise the biuret impurity of formula (A) in an amount of at least 0.1% by weight. In some embodiments, the particles comprise the biuret impurity of formula (A) in an amount of at least 0.3% by weight. In some embodiments, the particles comprise the biuret impurity of formula (A) in an amount of at most 0.7% by weight. In some embodiments, the particles comprise the biuret impurity of formula (A) in an amount of at most 0.5% by weight. In some embodiments, the particles comprise the biuret impurity of formula (A) in an amount of at most 0.3% by weight. In some embodiments, the particles comprise the biuret impurity of formula (A) in an amount of at most 0.2% by weight. In some embodiments, the particles comprise the biuret impurity of formula (A) in an amount of from 0% to about 0.3% by weight. In some embodiments, the particles comprise the biuret impurity of formula (A) in an amount of from 0% to about 0.05% by weight. In some embodiments, the particles comprise the biuret impurity of formula (A) in an amount of from 0% to about 0.03% by weight. In some embodiments, the weight percentage is relative to the total weight of ensifentrine (e.g., free base). In some embodiments, the weight percentage is relative to the total weight of the particles.

In some embodiments, the particles comprise 1-(2-(9-hydroxy-2-(mesitylimino)-10-methoxy-4-oxo-6,7-dihydro-2H-pyrimido[6,1-a]isoquinolin-3(4H)-yl)ethyl)urea (9-des-methyl impurity). In some embodiments, the particles comprise 1-(2-(10-hydroxy-2-(mesitylimino)-9-methoxy-4-oxo-6,7-dihydro-2H-pyrimido[6,1-a]isoquinolin-3(4H)-yl)ethyl)urea (10-des-methyl impurity). In some embodiments, the particles comprise the 9-des-methyl impurity and the 10-des-methyl impurity. In some embodiments, the particles comprise the 9-des-methyl or the 10-des-methyl impurity.

In some embodiments, the 9-des-methyl impurity has the structure:

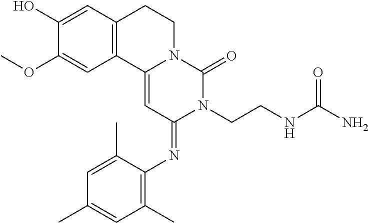 [SMILES: COc1cc2c(cc1O)CCn1c-2c/c(=N\c2c(C)cc(C)cc2C)n(CCNC(N)=O)c1=O]

In some embodiments, the 10-des-methyl impurity has the structure:

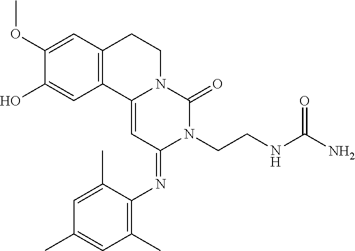 [SMILES: COc1cc2c(cc1O)-c1c/c(=N\c3c(C)cc(C)cc3C)n(CCNC(N)=O)c(=O)n1CC2]

In some embodiments, the (e.g., ensifentrine comprising) particles comprise the 9-des-methyl impurity in an amount of from 0% to about 0.1% by weight. In some embodiments, the particles comprise the 9-des-methyl impurity in an amount of at least 0.01% by weight. In some embodiments, the particles comprise the 9-des-methyl impurity in an amount of at least 0.05% by weight. In some embodiments, the particles comprise the 9-des-methyl impurity in an amount of at least 0.1% by weight. In some embodiments, the particles comprise the 9-des-methyl impurity in an amount of at most 0.2% by weight. In some embodiments, the particles comprise the 9-des-methyl impurity in an amount of 0.1% by weight. In some embodiments, the particles comprise the 9-des-methyl impurity in an amount of from about 0.01% to about 0.1% by weight. In some embodiments, the particles comprise the 9-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the particles comprise the 10-des-methyl impurity in an amount of from 0% to about 0.1% by weight. In some embodiments, the particles comprise the 10-des-methyl impurity in an amount of at least 0.01% by weight. In some embodiments, the particles comprise the 10-des-methyl impurity in an amount of at least 0.05% by weight. In some embodiments, the particles comprise the 10-des-methyl impurity in an amount of at least 0.1% by weight. In some embodiments, the particles comprise the 10-des-methyl impurity in an amount of at most 0.2% by weight. In some embodiments, the particles comprise the 10-des-methyl impurity in an amount of 0.1% by weight. In some embodiments, the particles comprise the 10-des-methyl impurity in an amount of from about 0.01% to about 0.1% by weight. In some embodiments, the particles comprise the 10-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the particles comprise (E)-2-(mesitylimino)-9,10-dimethoxy-2,3,6,7-tetrahydro-4H-pyrimido[6,1-a]isoquinolin-4-one (compound (I)). In some embodiments, the (e.g., ensifentrine) particles comprise (E)-3-(2-aminoethyl)-2-(mesitylimino)-9,10-dimethoxy-2,3,6,7-tetrahydro-4H-pyrimido[6,1-a]isoquinolin-4-one (compound (IV)). In some embodiments, the particles comprise both compound (I) and compound (IV). In some embodiments, the particles comprise compound (I) or compound (IV).

In some embodiments, the structure of compound (I) is:

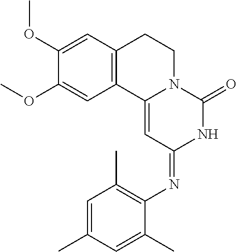 [SMILES: COc1cc2c(cc1OC)-c1c/c(=N\c3c(C)cc(C)cc3C)[nH]c(=O)n1CC2]

In some embodiments, the structure of compound (IV) is:

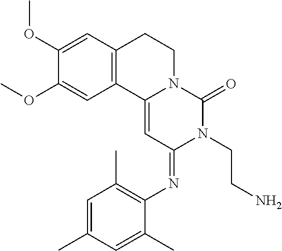 [SMILES: COc1cc2c(cc1OC)-c1c/c(=N\c3c(C)cc(C)cc3C)n(CCN)c(=O)n1CC2]

In some embodiments, the particles comprise from 0% to about 0.04% by weight of compound (I). In some embodiments, the particles comprise at least 0.01% by weight of compound (I). In some embodiments, the particles comprise at least 0.02% by weight of compound (I). In some embodiments, the particles comprise at most 0.05% by weight of compound (I). In some embodiments, the particles comprise at most 0.04% by weight of compound (I). In some embodiments, the (e.g., ensifentrine) particles comprise from 0% to about 0.04% by weight of compound (IV). In some embodiments, the particles comprise at least 0.01% by weight of compound (IV). In some embodiments, the particles comprise at least 0.02% by weight of compound (IV). In some embodiments, the particles comprise at most 0.05% by weight of compound (IV). In some embodiments, the particles comprise at most 0.04% by weight of compound (IV). In some embodiments, the particles do not comprise compound (I). In some embodiments, the particles do not comprise compound (IV). In some embodiments, the particles do not comprise compound (I) or compound (IV). In some embodiments, the particles comprise from 0% to about 0.04% by weight total of compound (I) and compound (IV). In some embodiments, the particles comprise at least 0.01% by weight total of compound (I) and compound (IV). In some embodiments, the particles comprise at least 0.02% by weight total of compound (I) and compound (IV). In some embodiments, the particles comprise at most 0.05% by weight total of compound (I) and compound (IV). In some embodiments, the particles comprise at most 0.04% by weight total of compound (I) and compound (IV). In some embodiments, the weight percentage is relative to the total weight of ensifentrine (e.g., free base). In some embodiments, the weight percentage is relative to the total weight of the particles.

In some embodiments, the BMIQU, biuret impurity, 9-des-methyl impurity, 10-des-methyl impurity, compound (I), and compound (IV), if present, may optionally be present in the form of a salt. In certain embodiments, the BMIQU, biuret impurity, 9-des-methyl impurity, 10-des-methyl impurity, compound (I), and compound (IV) are present in free base form.

In some embodiments, the particles provided herein comprise ensifentrine (e.g., ensifentrine free base (e.g., in crystalline Form I)), BMIQU, biuret impurity, 9-des-methyl impurity, and 10-des-methyl impurity. In some embodiments, the (e.g., ensifentrine) particles provided herein consists of ensifentrine (e.g., ensifentrine free base (e.g., in crystalline Form I)), BMIQU, biuret impurity, 9-des-methyl impurity, and 10-des-methyl impurity.

In some embodiments, the particles provided herein comprise from about 99.4% to about 99.9% by weight of ensifentrine, from about 0.01% to about 0.3% by weight of BMIQU, from 0% to about 0.1% by weight of the biuret impurity, from about 0.01% to about 0.2% by weight of the 9-des-methyl impurity, and from about 0.01% to about 0.2% by weight of the 10-des-methyl impurity, wherein the weight percent is relative to the total weight of the particles.

In some embodiments, the particles provided herein comprise from about 99.5% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

In some embodiments, the particles provided herein consists of from about 99.6% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

### Formulation

In some embodiments, the concentration of particles comprising the compound in the inhalable pharmaceutical composition is from 0.1 to 5.0 mg/mL, preferably from 0.1 to 2.5 mg/mL, more preferably from 1.0 to 2.0 mg/mL. In some embodiments, the concentration of particles comprising the compound in the inhalable pharmaceutical composition is about 1.2 mg/mL.

In some embodiments, the inhalable pharmaceutical composition further comprises one or more tonicity adjusters, one or more buffers and one or more surfactants. In some embodiments, the pharmaceutical composition comprises one or more tonicity adjusters. In some embodiments, the pharmaceutical composition comprises one or more buffers. In some embodiments, the pharmaceutical composition comprises one or more surfactants.

In some embodiments, the pharmaceutical compositions provided herein further comprise one or more tonicity adjusters. In some embodiments, the tonicity adjuster is sodium chloride. In some embodiments, the tonicity adjuster (e.g., sodium chloride) is present in the pharmaceutical composition at a concentration of at least 1 mg/mL (e.g., 2 mg/mL, 3 mg/mL, 4 mg/mL, 5 mg/mL, 6 mg/mL, 7 mg/mL). In some embodiments, the tonicity adjuster (e.g., sodium chloride) is present in the pharmaceutical composition at a concentration of at most 15 mg/mL (e.g., 14 mg/mL, 13 mg/mL, 12 mg/mL, 11 mg/mL, 10 mg/mL, 9 mg/mL). In some embodiments, the tonicity adjuster (e.g., sodium chloride) is present in the pharmaceutical composition at a concentration of from about 1 mg/mL to about 15 mg/mL, about 3 mg/mL to about 10 mg/mL, about 4 mg/mL to about 10 mg/mL, about 5 mg/mL to about 11 mg/mL, about 5 mg/mL to about 9 mg/mL, or about 6 mg/mL to about 9 mg/mL. In some embodiments, the tonicity adjuster (e.g., sodium chloride) is present in the pharmaceutical composition at a concentration of about 7 mg/mL to about 10 mg/mL. In some embodiments, the tonicity adjuster (e.g., sodium chloride) is present in the pharmaceutical composition at a concentration of 8 mg/mL to 9 mg/mL. In certain embodiments, the tonicity adjuster (e.g., sodium chloride) is present in the pharmaceutical composition at a concentration of (e.g., about) 8.6 mg/mL.

In some embodiments, the pharmaceutical compositions provided herein further comprise one or more buffers. Examples of buffers include a citrate buffer, a phosphate buffer, an acetate buffer, and a bicarbonate buffer. Preferably, the buffer is a phosphate buffer, for instance sodium dihydrogen phosphate dihydrate and/or disodium phosphate dihydrate. In certain embodiments, the buffer is a phosphate buffer. In some embodiments, the buffer comprises sodium dihydrogen phosphate dihydrate and/or disodium hydrogen phosphate dihydrate. In some embodiments, the buffer (e.g., sodium dihydrogen phosphate dihydrate and/or disodium hydrogen phosphate dihydrate) is present in the pharmaceutical composition at a concentration of at least 0.1 mg/mL (e.g., 0.2 mg/mL, 0.4 mg/mL, 0.8 mg/mL, 1 mg/mL, or 1.4 mg/mL). In some embodiments, the buffer (e.g., sodium dihydrogen phosphate dihydrate and/or disodium hydrogen phosphate dihydrate) is present in the pharmaceutical composition at a concentration of 4 mg/mL (e.g., 3.8 mg/mL, 3.3 mg/mL, 2.8 mg/mL, 2.5 mg/mL, or 2 mg/mL). In some embodiments, the buffer (e.g., sodium dihydrogen phosphate dihydrate and/or disodium hydrogen phosphate dihydrate) is present in the pharmaceutical composition at a concentration of about 1 mg/mL to about 2 mg/mL, about 1.2 mg/mL to about 1.8 mg/mL, or about 1.4 mg/mL to about 1.7 mg/mL. In some embodiments, the buffer (e.g., sodium dihydrogen phosphate dihydrate and/or disodium hydrogen phosphate dihydrate) is present in the pharmaceutical composition at a concentration of about 1.5 mg/mL to about 1.7 mg/mL.

In some embodiments, the pharmaceutical composition comprises sodium dihydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL). In some embodiments, the pharmaceutical composition comprises sodium dihydrogen phosphate dihydrate at a concentration of at most 1.2 mg/mL (e.g., 1.1 mg/mL, 1.0 mg/mL, 0.9 mg/mL, 0.8 mg/mL). In some embodiments, the pharmaceutical composition comprises sodium dihydrogen phosphate dihydrate at a concentration of from about 0.5 mg/mL to about 0.9 mg/mL. In some embodiments, the pharmaceutical composition comprises sodium dihydrogen phosphate dihydrate at a concentration of about 0.7 mg/mL to about 0.8 mg/mL. In some embodiments, the pharmaceutical composition comprises sodium dihydrogen phosphate dihydrate at a concentration of about 0.7 mg/mL. In certain embodiments, the pharmaceutical composition comprises sodium dihydrogen phosphate dihydrate at a concentration of (e.g., about) 0.744 mg/mL.

In some embodiments, the pharmaceutical composition comprises disodium hydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL, 0.7 mg/mL). In some embodiments, the pharmaceutical composition comprises disodium hydrogen phosphate dihydrate at a concentration of at most 1.2 mg/mL (e.g., 1.1 mg/mL, 1.0 mg/mL, 0.9 mg/mL). In some embodiments, the pharmaceutical composition comprises disodium hydrogen phosphate dihydrate at a concentration of from about 0.7 mg/mL to about 1 mg/mL. In some embodiments, the pharmaceutical composition comprises disodium hydrogen phosphate dihydrate at a concentration of about 0.8 mg/mL to about 0.9 mg/mL. In some embodiments, the pharmaceutical composition comprises disodium hydrogen phosphate dihydrate at a concentration of about 0.8 mg/mL. In some embodiments, the pharmaceutical composition comprises disodium hydrogen phosphate dihydrate at a concentration of about 0.9 mg/mL. In certain embodiments, the pharmaceutical composition comprises disodium hydrogen phosphate dihydrate at a concentration of (e.g., about) 0.853 mg/mL.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

In some embodiments, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). In some embodiments, the sterile liquid vehicle comprises polysorbate 20 and/or sorbitan monolaurate (Span 20).

In some embodiments, the pharmaceutical composition comprises the one or more surfactants (e.g., polysorbate 20 and/or sorbitan monolaurate) in a concentration of at least 0.05 mg/mL (e.g., 0.1 mg/mL, 0.2 mg/mL, 0.3 mg/mL, 0.4 mg/mL). In some embodiments, the pharmaceutical composition comprises the one or more surfactants (e.g., polysorbate 20 and/or sorbitan monolaurate) in a concentration of at most 1 mg/mL (e.g., 0.9 mg/mL, 0.8 mg/mL, 0.7 mg/mL, 0.6 mg/mL). In some embodiments, the pharmaceutical composition comprises the one or more surfactants (e.g., polysorbate 20 and/or sorbitan monolaurate) in a concentration of from about 0.3 mg/mL to about 0.7 mg/mL. In some embodiments, the pharmaceutical composition comprises the one or more surfactants (e.g., polysorbate 20 and/or sorbitan monolaurate) at a concentration of from about 0.01 mg/mL to 2 mg/mL. In some embodiments, the pharmaceutical composition comprises the one or more surfactants (e.g., polysorbate 20 and/or sorbitan monolaurate) in a concentration of from about 0.4 mg/mL to about 0.6 mg/mL. In some embodiments, the pharmaceutical composition comprises the one or more surfactants (e.g., polysorbate 20 and/or sorbitan monolaurate) at a concentration of about 0.5 mg/mL. In some embodiments, the pharmaceutical composition comprises the one or more surfactants (e.g., polysorbate 20 and/or sorbitan monolaurate) at a concentration of about 0.6 mg/mL. In certain embodiments, the pharmaceutical composition comprises the one or more surfactants (e.g., polysorbate 20 and/or sorbitan monolaurate) in a concentration of (e.g., about) 0.55 mg/mL.

In some embodiments, the pharmaceutical composition comprises polysorbate 20 (e.g., Tween 20) in a concentration of at least 0.1 mg/mL (e.g., 0.2 mg/mL, 0.3 mg/mL, 0.4 mg/mL). In some embodiments, the pharmaceutical composition comprises polysorbate 20 (e.g., Tween 20) in a concentration of at most 1 mg/mL (e.g., 0.9 mg/mL, 0.8 mg/mL, 0.7 mg/mL, 0.6 mg/mL). In some embodiments, the pharmaceutical composition comprises polysorbate 20 (e.g., Tween 20) in a concentration of from about 0.3 mg/mL to about 0.7 mg/mL. In some embodiments, the pharmaceutical composition comprises polysorbate 20 (e.g., Tween 20) in a concentration of about 0.4 mg/mL to about 0.6 mg/mL. In certain embodiments, the pharmaceutical composition comprises polysorbate 20 (e.g., Tween 20) in a concentration of about 0.5 mg/mL.

In some embodiments, the pharmaceutical composition comprises sorbitan monolaurate (Span 20) in a concentration of at least 0.01 mg/mL (e.g., 0.02 mg/mL, 0.03 mg/mL, 0.04 mg/mL). In some embodiments, the pharmaceutical composition comprises sorbitan monolaurate (Span 20) in a concentration of at most 0.1 mg/mL (e.g., 0.09 mg/mL, 0.08 mg/mL, 0.07 mg/mL, 0.06 mg/mL). In some embodiments, the pharmaceutical composition comprises sorbitan monolaurate (Span 20) in a concentration of from about 0.03 mg/mL to about 0.07 mg/mL. In some embodiments, the pharmaceutical composition comprises sorbitan monolaurate (Span 20) in a concentration of from about 0.04 mg/mL to about 0.06 mg/mL. In certain embodiments, the pharmaceutical composition comprises sorbitan monolaurate (Span 20) in a concentration of (e.g., about) 0.05 mg/mL.

For instance, a method disclosed herein may comprise administering to the patient an inhalable liquid pharmaceutical composition comprising:
water;
particles consisting of ensifentrine free base at a concentration of from 0.1 to 20 mg/mL;
one or more tonicity adjusters at a total concentration of from 1.0 to 15 mg/mL;
one or more buffers at a total concentration of from 0.1 to 4 mg/mL; and
one or more surfactants at a total concentration of from 0.05 to 3 mg/mL.

The inhalable liquid pharmaceutical composition may comprise:
water;
particles consisting of ensifentrine free base at a concentration of from 0.5 to 6 mg/mL;
sodium chloride at a concentration of from 5 to 12 mg/mL;
sodium dihydrogen phosphate dihydrate at a concentration of from 0.3 to 2 mg/mL;
disodium phosphate dihydrate at a concentration of from 0.3 to 2 mg/mL;
polysorbate 20 at a concentration of from 0.1 to 1.5 mg/mL; and
sorbitan monolaurate at a concentration of from 0.01 to 0.5 mg/mL.

The inhalable liquid pharmaceutical composition may comprise:
water;
particles consisting of ensifentrine free base at a concentration of 1.2 mg/mL;
sodium chloride at a concentration of from 8.6 mg/mL;
sodium dihydrogen phosphate dihydrate at a concentration of 0.744 mg/mL;
disodium phosphate dihydrate at a concentration of from 0.853 mg/mL;
polysorbate 20 at a concentration of 0.5 mg/mL; and
sorbitan monolaurate at a concentration of 0.05 mg/mL.

In some embodiments, the pharmaceutical compositions herein are provided in an ampule, such as for administration by inhalation using a nebulizer. In some embodiments, the ampule comprises the suspension of ensifentrine particles as described herein. In some embodiments, the ampule comprises about 2.6 mL of the pharmaceutical composition. In some embodiments, the ampule comprises at least 3 mg of ensifentrine (e.g., ensifentrine free base). In some embodiments, the ampule comprises about 3.1 mg of ensifentrine. In some embodiments, the ampule is a low density polyethylene (LDPE) ampule.

### Combinations

In some embodiments, the compound may be used as disclosed herein in combination with a second active agent. The compound may be administered separately, consecutively, sequentially or simultaneously with the second active agent. The patient may already be taking a second active agent as a background therapy for COPD or a condition disclosed herein. Alternatively, treatment with the second active agent may start at around or at the same time as treatment with the compound. In some embodiments, the compound and the second active agent may be administered in a fixed combination.

In some embodiments, the second active agent is a muscarinic receptor antagonist or a beta-adrenergic receptor agonist. In some embodiments, the compound may accordingly be used in combination with a muscarinic receptor antagonist or a beta-adrenergic receptor agonist. In some embodiments, the second active agent may be a long-acting muscarinic receptor antagonist (LAMA) or a long-acting beta-adrenergic receptor agonist (LABA).

Examples of LAMAs include aclidinium, darotropium, tiotropium, glycopyrrolate and umeclidinium. Examples of LABAs include salmeterol, formoterol, indacaterol, vilanterol, olodaterol, abediterol and carmoterol. In some embodiments, the patient may be using a beta-agonist (for instance salbutamol) as a rescue medication.

In other embodiments, the compound may be used in absence of a second agent. In some embodiments the compound may be used in absence of a LAMA, LABA, ICS, or a combination thereof. In some embodiments, the compound may be used in absence of a LAMA. In some embodiments, the compound may be used in absence of a LABA. In some embodiments, the compound may be used in absence of an ICS. In some embodiments, the subject is not concurrently taking a LAMA, LABA, ICS, or a combination thereof while being administered the compound.

### Kits

Also provided herein in some embodiments are kits comprising a compound or pharmaceutical composition provided herein. In some embodiments, the kit comprises ensifentrine, such as ensifentrine free base.

In some embodiments, the kit comprises a nebuliser for administration of ensifentrine.

In some embodiments, the kit comprises instructions. In some embodiments, the instructions are for treating are for treating COPD in a patient with a blood eosinophil count of less than 300 cells/μL, for example less than 150 cells/μL or less than 100 cells/μL. In some embodiments, the instructions are for treating COPD in a patient with a blood eosinophil count of less than 150 cells/μL. In some embodiments, the instructions are for treating COPD in a patient with a blood eosinophil count of less than 100 cells/μL.

In some embodiments, the instructions are for treating COPD exacerbations in a patient with a blood eosinophil count of less than 300 cells/μL. In some embodiments, the instructions are for treating COPD exacerbations in a patient with a blood eosinophil count of less than 150 cells/μL. In some embodiments, the instructions are for treating COPD exacerbations in a patient with a blood eosinophil count of less than 100 cells/μL.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the invention described herein may be employed in practicing the disclosure. It is intended that the following claims define the scope of the disclosure and that methods and structures within the scope of these claims and their equivalents be covered thereby.

### EXAMPLES

### Example 1: Subjects with Blood Eosinophil Counts of Less than 150 Cells/μL

### Study Design

A clinical study was conducted to determine the efficacy of ensifentrine in treating COPD compared with placebo. Ensifentrine was administered by nebuliser at a dose of 3 mg twice daily (BID) for 24 weeks. The study was a multi-centre, randomized, double-blind, parallel-group, placebo-controlled trial with around 800 patients and 5:3 randomization.

The study population included patients aged 40-80 years with moderate to severe COPD (FEV₁ 30%-70% p.n., FEV₁/forced vital capacity (FVC) ratio <0.7, with mMRC≥2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC)0-12 h post-dose at week 12. Secondary endpoints of the study included: frequency of moderate/severe COPD exacerbations over 24 Weeks; time to first moderate/severe COPD exacerbation; and effect of blood eosinophil count of patients.

### Methods

COPD severity is derived as follows: mild: 80%<=FEV₁, moderate: 50%<=FEV₁<80% predicted, severe: 30%<=FEV₁<50% predicted and very severe: FEV₁<30% predicted, post bronchodilator dose at Screening.

Baseline FEV₁ is the mean of the two measurements taken before study medication on the day of first dosing, i.e. <40 minutes and just prior to dosing, both pre-dose on day 1.

Average FEV₁ AUC0-12 h is defined as area under the curve over 12 hours of the FEV₁, divided by 12 hours.

Exacerbations were defined as worsening of 2 or more major symptoms (dyspnea, sputum volume, and sputum purulence) or worsening of any 1 major symptom together with any 1 of the following minor symptoms: sore throat, colds, fever without other cause, and increased cough or wheeze for at least 2 consecutive days. COPD exacerbations were considered to be of moderate severity if treatment with systemic corticosteroids and/or antibiotics was required and were considered to be severe if hospitalization was required.

The blood eosinophil count of patients was determined as follows.
A blood sample (2 mL) was collected in 2 mL EDTA tube and mixed immediately using gentle inversion 8-10 times.
Eosinophils were tested as part of the Complete Blood Count (CBC) test panel and are part of the White Blood Cell (WBC) differential.
Testing was performed on the Beckman Coulter DxH 800, with testing performed daily on ambient samples.
The haematology parameters that constitute the CBC panel were produced using methods and technologies within the DxH800, including Coulter principle and VCSn laser flow cell technology.
The WBC differential was obtained using VCSn technology (Volume, Conductivity and Scatter) based on simultaneous measuring of cell volume, high frequency conductivity and laser light scatter. This produced an automated WBC differential.
During analysis, if any parameter was abnormal or outside established limits the instrument flagged this as abnormal and produced a stained blood slide.
Blood film review was completed using manual microscopy and a technician confirmed any abnormal findings.
The technician could have also provided a manual WBC differential during blood film review or confirm the automated WBC differential.
Finalised results were then provided.

### Formulation

The investigational product and placebo were provided in 2.5 mL unit dose format in an ampule and administered via a nebuliser. The formulation of the investigational product (ensifentrine suspension formulation) and placebo are shown in Table 1 below.

TABLE 1

| Constituent | Concentration (mg/mL) |
|---|---|
| Ensifentrine particles (RPL554) | 1.2 (for the active) |
| | or 0 (for Placebo) |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

### Results

The primary endpoint of average FEV₁ (AUC)0-12 h at Week 12 was met. All subgroups showed improvement in lung function with ensifentrine that was statistically significant. The results are shown in Table 2.

TABLE 2

| Average FEV₁ (AUC)0-12 h | | |
|---|---|---|
| post-dose at Week 12: | Ensifentrine 3 mg | Placebo |
| Change from Baseline (CFB) | (n = 498) | (n = 291) |
| Observed N | 424 | 231 |
| Mean Baseline FEV₁, L | 1.3323 | 1.2316 |
| CFB Least Squares Mean, | 48.2 (30.0, 66.5) | −45.7 (−69.7, −21.6) |
| mL (95% CI) | | |
| Placebo-corrected CFB LS | 93.9 (64.5, 123.3) | — |
| Mean Diff, mL (95% CI) | | |
| p-value | <0.0001 | — |

Ensifentrine treatment resulted in approximately 40% reduction in relative risk of moderate or severe COPD exacerbation vs placebo (including 52% of patients on background bronchodilator medications).

In a subgroup analysis, the reduction in COPD exacerbations was observed in patients with a blood eosinophil count of <300 cells/μL. In particular, a reduction of around 50% in relative risk of moderate or severe COPD exacerbation vs placebo was observed in this subgroup.

In another subgroup analysis, the reduction in COPD exacerbations was observed in patients with a blood eosinophil count of <150 cells/μL, similar to that for patients with blood eosinophil count of >150 cells/μL, as shown in Table 3.

TABLE 3

| | Rate Ratio (95% | Exacerbation |
|---|---|---|
| | Confidence Interval) | Rate Reduction |
| Blood eosinophils > 150 | 0.57 (0.34, 0.97) | 43% |
| cells/μL (n = 445) | | |
| Blood eosinophils ≤ 150 | 0.58 (0.30, 1.14) | 42% |
| cells/μL (n = 343) | | |

The efficacy in reducing COPD exacerbations was also observed in patients with an even lower blood eosinophil count of <150 cells/μL. In particular, a reduction of around 40% in relative risk of moderate or severe COPD exacerbation vs placebo was observed in this subgroup. Safety assessments confirmed that there was no significant increase in the risk of pneumonia in patients with a blood eosinophil count of <300 cells/μL following administration of ensifentrine.

Ensifentrine was accordingly found to be an effective treatment for COPD, including a reduction in risk of COPD exacerbations, in patients with a blood eosinophil count of <300 cells/μL.

Comparison with LAMA-LABA-ICS Treatment

Suissa (“Single-inhaler triple versus dual bronchodilator therapy for GOLD E and other exacerbating patients with COPD: Real-world comparative effectiveness and safety”, European Respiratory Journal 2023) reports an analysis of single-inhaler triple (LAMA-LABA-ICS) versus dual (LAMA-LABA) bronchodilator therapy COPD patients, stratified by blood eosinophil count. (LAMA=long-acting muscarinic antagonist; LABA=long-acting beta2-agonist; ICS=inhaled corticosteroid.)

In patients with a blood eosinophil count of 300 cells/μL, triple (LAMA-LABA-ICS) therapy was found to be effective, and associated with a reduction in moderate or severe COPD exacerbations.

In contrast, for patients with a blood eosinophil count of <300 cells/μL, triple (LAMA-LABA-ICS) therapy was no more effective than a dual (LAMA-LABA) single-inhaler at reducing the incidence of a moderate or severe exacerbation, showing only a 6% reduction in risk of a moderate or severe exacerbation compared with the dual therapy.

Furthermore, the triple therapy resulted in a 43% increase in the risk of severe pneumonia leading to hospitalization in the patients with a blood eosinophil count of <300 cells/μL.

It was therefore found that the inclusion of an anti-inflammatory agent (ICS) was associated with a significant increase in the risk of severe pneumonia in patients with a blood eosinophil count of <300 cells/μL, while only offering a marginal decrease in moderate to severe COPD exacerbation risk. Treatment with triple (LAMA-LABA-ICS) therapy in this patient group was therefore not recommended.

### Conclusion

It has been found that ensifentrine provides a combined anti-inflammatory and bronchodilator therapy which may be used safely and effectively to treat COPD in patients with a blood eosinophil count of <300 cells/μL.

### Example 2: Subjects with Blood Eosinophil Counts of Less than 100 or 300 Cells/μL

### Methods

This analysis pooled data over 24 weeks from the modified intent-to-treat (mITT) populations of two phase 3, global, multicenter, randomized, double-blind, parallel-group, placebo-controlled clinical trials in patients with symptomatic, moderate-to-severe COPD. Eligible patients had moderate-to-severe COPD (forced expiratory volume in the first second [FEV₁]/forced vital capacity <0.7, post-bronchodilator 30%-70% of predicted normal FEV₁), were aged 40-80 years, and were symptomatic at randomization (score ≥2 on the Modified Medical Research Council scale). Patients with asthma or a history of life-threatening COPD, recent hospitalizations for COPD, pneumonia, COVID-19 within 12 weeks of the trial, or COPD exacerbation requiring oral or intravenous steroids within 3 months of the trial were excluded. There was no requirement for prior history of exacerbations for eligibility. Eligible patients were randomly assigned 5:3 to receive 3 mg ensifentrine BID or placebo over 24 weeks via a standard jet nebulizer (PARI, Germany); a subset of patients in one clinical trial received treatment for 48 weeks. Randomization in said clinical trial was stratified by stable background maintenance with LAMA or LABA (yes or no), smoking status (current or former), and treatment duration (24 or 48 weeks); randomization in the second clinical trial was stratified by stable background maintenance with LAMA or LABA (yes or no) and smoking status (current or former). Rates of moderate/severe exacerbation and time to first exacerbation event were evaluated at 24 weeks. Moderate COPD exacerbations were defined as worsening of COPD symptoms (≥2 major symptoms or 1 major and 1 minor) for ≥2 days, requiring 3 days or more of therapy with oral or systemic corticosteroids and/or antibiotics, whereas severe COPD exacerbations required worsening of specified symptoms and in-patient hospitalization. Major symptoms included dyspnea, sputum volume, and sputum purulence (color). Minor symptoms included sore throat, colds (nasal discharge and/or nasal congestion), fever (oral temperature >37.5° C.) without other cause, increased cough, or increased wheeze.

A post hoc analysis was conducted to determine the effect of ensifentrine versus placebo in reducing the risk of transitioning from a lower exacerbation risk group (infrequent exacerbators), analogous to Global Initiative for Chronic Obstructive Lung Disease (GOLD) group B, to a frequent exacerbator status, analogous to GOLD group E. Group E defines a group of patients at substantially higher risk of future exacerbations compared with group B. According to the GOLD 2024 Strategy, patients with COPD categorized as group B are symptomatic (Modified Medical Research Council scale ≥2 or COPD assessment test ≥10), experience 0 or 1 moderate exacerbations (not leading to hospitalization) in the prior year, while group E includes patients who experience 2 or more exacerbations, or an exacerbation leading to hospitalization. GOLD 2024 group B to group E served as the basis for this analysis of risk of transitioning from low exacerbation risk to high risk. The transition events were defined as follows: (1) there was a pre-treatment exacerbation and a post-treatment exacerbation and the pre-treatment exacerbation was within 1 year of the post-treatment exacerbation and the post-treatment exacerbation occurred within 24 weeks of first dose of study medication; (2) there were 2 post-treatment exacerbations that occurred within 24 weeks of first dose of study medication; or (3) an exacerbation occurred within 24 weeks of the first dose of study medication and hospitalization due to exacerbation was recorded.

A post-hoc subgroup analysis was performed to assess the effect of ensifentrine treatment on exacerbation rate and risk (time to first exacerbation) according to clinical characteristics and known risk factors for COPD exacerbations, including blood eosinophil count at screening (≥100 or <100 and ≥300 or <300 cells/μL).

The moderate/severe exacerbation rate was compared between treatment groups by using a negative binomial model with adjustment for treatment, region, trial, background medication strata, and smoking status, with log(trial time) (years) as an offset. Time to first moderate/severe exacerbation was analyzed by using the log-rank test, stratified by region, trial, background medication strata, and smoking status. Withdrawn patients without any events were censored at last day in study. Missing data were not imputed. Proportionality of the hazard was tested by the interaction between treatment and log(trial time). A Cox proportional hazards model was used with the same factors to estimate the effect as a hazard ratio. All subgroup analyses omitted region as a factor. A Cox proportional hazards regression model, adjusted for treatment, trial, background medication strata, and smoking status, was used to evaluate the duration of time until a GOLD 2024 group B to group E transition event.

### Results

This pooled analysis included 1549 patients, comprising 975 ensifentrine-treated and 574 placebo-treated patients. The demographics and baseline disease characteristics of the pooled populations were similar, and consistent with each individual trial. At the time of initial screening, 22.6% of patients receiving ensifentrine and 23.7% of those receiving placebo reported a COPD exacerbation in the preceding 15 months. 62% of patients were on concomitant bronchodilator therapy (LAMA or LABA) and 18% were on concomitant ICS therapy during the trials.

In the pooled analysis, treatment with 3 mg ensifentrine BID over 24 weeks was associated with a 41% reduction in the moderate/severe exacerbation rate compared with placebo (rate ratio [RR], 0.59; 95% CI, 0.43-0.80; P<0.001). The time to first moderate/severe exacerbation over 24 weeks was also significantly delayed compared with placebo (log rank test P=0.001). Furthermore, a Cox proportional hazards model demonstrated a significant 41% reduction in risk (hazard ratio [HR], 0.59; 95% CI, 0.44-0.81; P<0.001). Proportionality of hazards was assessed graphically and by testing the interaction treatment by log(trial time) that was not significant. Based on the exacerbation rate reduction, the number needed to treat (NNT) for the prevention of 1 exacerbation annually is equal to 6.25 patients.

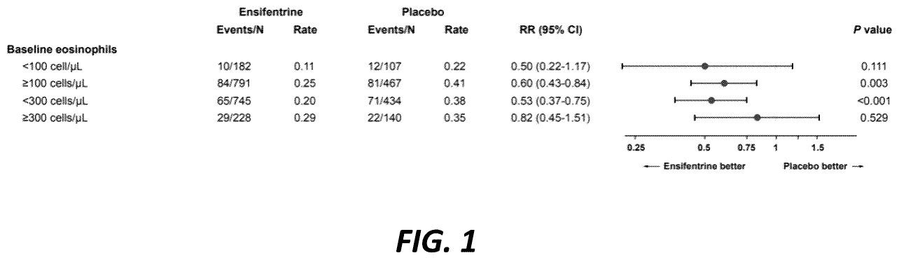

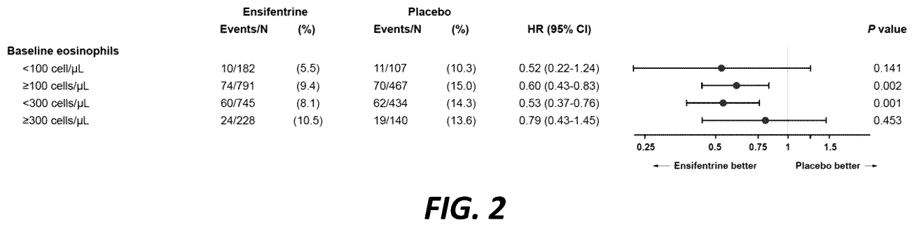

Patients with blood eosinophil counts 100 cells/μL and <300 cells/μL who were treated with ensifentrine demonstrated a consistent reduction in the rate and risk of exacerbations. Numerical reductions were observed in patients with blood eosinophil counts <100 cells/μL and ≥300 cells/μL (as shown in Tables 4-7 and FIG. 1 and FIG. 2). Subjects with blood eosinophil counts of <100 cells/μL demonstrated reductions in exacerbation rate of about 50%. Subjects with blood eosinophil counts of ≤300 cells/μL demonstrated reductions in exacerbation rate of about 47%.

TABLE 4

| | Hazard Ratio (95% |
|---|---|
| | Confidence Interval) |
| Blood eosinophils ≥ 300 | 0.79 (0.43, 1.45) |
| cells/μL (n = 24) | |
| Blood eosinophils < 300 | 0.53 (0.37, 0.76) |
| cells/μL (n = 60) | |

TABLE 5

| | Rate Ratio (95% Confidence |
|---|---|
| | Interval) |
| Blood eosinophils ≥ 300 | 0.82 (0.45, 1.51) |
| cells/μL (n = 29) | |
| Blood eosinophils < 300 | 0.53 (0.37, 0.75) |
| cells/μL (n = 65) | |

TABLE 6

| | Hazard Ratio (95% |
|---|---|
| | Confidence Interval) |
| Blood eosinophils ≥ 100 | 0.60 (0.43, 0.83) |
| cells/μL (n = 74) | |
| Blood eosinophils < 100 | 0.52 (0.22, 1.24) |
| cells/μL (n = 10) | |

TABLE 7

| | Rate Ratio (95% Confidence |
|---|---|
| | Interval) |
| Blood eosinophils ≥ 100 | 0.60 (0.43, 0.84) |
| cells/μL (n = 74) | |
| Blood eosinophils < 100 | 0.50 (0.22, 1.17) |
| cells/μL (n = 10) | |

Treatment with ensifentrine numerically delayed the transition from lower to higher exacerbation risk (group B to group E) compared with patients who were treated with placebo.

### Conclusion

In this pooled analysis of both phase 3 trials, ensifentrine substantially and significantly reduced the rate and risk of moderate/severe exacerbations over 24 weeks across a population of patients with COPD. Treatment effects showing reductions in the rate and risk of exacerbations favoring ensifentrine over placebo were observed in all subgroups assessed. Reductions in exacerbation rate and risk (measured by time to first event) were observed in patients with blood eosinophils counts ≥100 cells/μL and <300 cells/μL with numerical reductions observed in patients with blood eosinophil counts <100 cells/μL and ≥300 cells/μL.

### Numbered Embodiments

1. A compound for use in a method of treating chronic obstructive pulmonary disease (COPD) in a patient, which compound is ensifentrine or a pharmaceutically acceptable salt thereof,
   wherein the patient has a blood eosinophil count of <300 cells/μL.
2. A compound for use according to embodiment 1, wherein the method of treating COPD comprises decreasing the frequency and/or severity of COPD exacerbations in the patient.
3. A compound for use according to embodiment 2, wherein the decreasing the frequency and/or severity of COPD exacerbations in the patient is compared to frequency of COPD exacerbations or severity of COPD exacerbations in an untreated subject having COPD and not administered the composition.
4. A compound for use according to any one of the preceding embodiments, wherein the method of treating COPD comprises decreasing the frequency of COPD exacerbations in the patient.
5. A compound for use according to any one of the preceding embodiments, wherein the method decreases the frequency of COPD exacerbations or the severity of COPD exacerbations in the subject by at least 20% compared to the frequency of COPD exacerbations or the severity of COPD exacerbations in the untreated subject having COPD and not administered the compound.
6. A compound for use according to any one of the preceding embodiments, wherein the method decreases the frequency of COPD exacerbations or the severity of COPD exacerbations in the subject by at least 40% compared to the frequency of COPD exacerbations or the severity of COPD exacerbations in the untreated subject having COPD and not administered the compound.
7. A compound for use according to any one of the preceding embodiments, wherein the method of treating COPD comprises increasing the time to first COPD exacerbation in the patient.
8. A compound for use according to any one of the preceding embodiments, wherein the patient is susceptible to COPD exacerbations.
9. A compound for use according to embodiment 8, wherein the patient susceptible to COPD exacerbations is suffering from one or more disease or condition selected from asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease or tuberculosis, optionally wherein the asthma is allergic asthma, steroid resistant asthma, severe asthma or paediatric asthma.
10. A compound for use according to any one of the preceding embodiments, wherein the patient has suffered one or more COPD exacerbations in the year preceding first administration of the compound, optionally wherein the patient has suffered two or more COPD exacerbations in the year preceding first administration of the compound.
11. A compound for use according to any one of the preceding embodiments, wherein the COPD exacerbations comprise one or more of dyspnea, increased coughing, increased sputum volume, sputum purulence, wheezing, sore throat, a cold, and fever.
12. A compound for use according to any one of the preceding embodiments, wherein the patient is susceptible to pneumonia.
13. A compound for use according to embodiment 12, wherein the patient susceptible to pneumonia is aged 65 years or older and/or has a BMI of at least 30.
14. A compound according to embodiment 12 or embodiment 13, wherein the patient susceptible to pneumonia is suffering from one or more disease or condition selected from diabetes, dementia, Parkinson's disease, cystic fibrosis, cancer, multiple sclerosis and osteoporosis, or wherein the patient susceptible to pneumonia has suffered a stroke or transient ischaemic attack.
15. A compound according to any one of the preceding embodiments, wherein the patient has a blood eosinophil count of <275 cells/μL and/or the patient has a blood eosinophil count of >160 cells/μL.
16. A compound according to any one of the preceding embodiments, wherein the patient has a blood eosinophil count of from 170 to 260 cells/μL.
17. A compound for use according to any one of embodiments 1 to 14, wherein the patient has a blood eosinophil count of <150 cells/μL.
18. A compound for use according to any one of embodiments 1 to 14, wherein the patient has a blood eosinophil count of <100 cells/μL.
19. A compound according to any one of the preceding embodiments, wherein the blood eosinophil count of the patient is as determined from a sample of the patient's blood using a Coulter counter.
20. A compound according to any one of the preceding embodiments, wherein the patient is not receiving an inhaled corticosteroid at the time of treatment with the compound.
21. A compound for use according to any one of the preceding embodiments, wherein the method comprises administering the compound to the patient by inhalation.
22. A compound for use according to any one of the preceding embodiments, wherein the method comprises administering the compound to the patient by inhalation by nebuliser.
23. A compound for use according to any one of the preceding embodiments, wherein the compound is ensifentrine.
24. A compound for use according to any one of the preceding embodiments, wherein the method comprises administering the compound to the patient once, twice or three times per day.
25. A compound for use according to any one of the preceding embodiments, wherein the method comprises administering the compound to the patient twice a day.
26. A compound for use according to any one of the preceding embodiments, wherein the method comprises administering a total daily dose of the compound of from 0.5 to 10 mg, preferably from 5 to 7 mg.
27. A compound for use according to any one of the preceding embodiments, wherein the method comprises administering the compound to the patient twice a day in a first dose of from 2 to 4 mg and a second dose of from 2 to 4 mg.
28. A compound for use according to any one of the preceding embodiments, wherein the method comprises administering a dose of about 3 mg the compound to the patient twice a day (3 mg BID), preferably wherein the method comprises administering by nebuliser a dose of about 3 mg the compound to the patient twice a day.
29. A compound for use according to any one of the preceding embodiments, wherein the method comprises administering the compound to the patient at least once per day for at least 8 weeks, preferably for at least 16 weeks, more preferably for at least 24 weeks.
30. A compound for use according to any one of the preceding embodiments, wherein the method comprising administering an inhalable pharmaceutical composition comprising a suspension of particles of the compound in a diluent.
31. A compound for use according to embodiment 30, wherein the inhalable pharmaceutical composition comprises, relative to the total weight of the inhalable pharmaceutical composition:
   (a) the particles (e.g., comprising ensifentrine) at a concentration of from 0.8 to 1.6 mg/mL;
   (b) one or more surfactants at a total concentration of from 0.1 to 1 mg/mL;
   (c) one or more buffers at a total concentration of from 1 to 2 mg/mL; and
   (d) water.
32. A compound for use according to embodiment 30 or 31, wherein the inhalable pharmaceutical composition comprises, relative to the total weight of the inhalable pharmaceutical composition:
   (a) the particles (e.g., comprising ensifentrine) at a concentration of from 1 to 1.4 mg/mL;
   (b) polysorbate 20 at a concentration of 0.3 to 0.7 mg/mL;
   (c) sorbitan monolaurate at a concentration of from 0 to 0.1 mg/mL;
   (d) sodium dihydrogen phosphate dihydrate at a concentration of from 0.5 to 1 mg/mL;
   (e) disodium hydrogen phosphate dihydrate at a concentration of from 0.5 to 1 mg/mL;
   (f) sodium chloride at a concentration of from 5 to 10 mg/mL; and
   (g) water.
33. A compound for use according to any one of embodiments 30 to 32, wherein the inhalable pharmaceutical composition comprises, relative to the total weight of the inhalable pharmaceutical composition:
   (a) the particles (e.g., comprising ensifentrine) at a concentration of 1.2 mg/mL;
   (b) polysorbate 20 at a concentration of 0.5 mg/ml;
   (c) sorbitan monolaurate at a concentration of 0.05 mg/ml;
   (d) sodium dihydrogen phosphate at a concentration of 0.744 mg/ml;
   (e) disodium hydrogen phosphate at a concentration of 0.853 mg/ml;
   (f) sodium chloride at a concentration of 8.6 mg/ml; and
   (g) water.
34. A compound for use according to any one of embodiments 30 to 33, wherein the particles comprise ensifentrine free base.
35. A compound for use according to any one of embodiments 30 to 34, wherein the particles comprise ensifentrine (e.g., free base) in an amount of at least 99 wt %, optionally at least 99 wt % relative to the total weight of the particles.
36. A compound for use according to any one of embodiments 30 to 35, wherein the particles have a Dv50 of from about 1.0 μm to about 2.2 μm.
37. A compound for use according to any one of embodiments 30 to 36, wherein the particles have a Dv10 of from about 0.3 μm to about 0.9 μm and/or a Dv90 of from about 2.3 μm to about 4.5 μm.
38. A compound for use according to any one of embodiments 30 to 37, wherein the particles comprise:
   from 99.4 to 99.9 wt % of ensifentrine (e.g., free base);
   from 0.01 wt % to 0.30 wt % of BMIQU;
   from 0.00 wt % to 0.10 wt % of the biuret impurity;
   from 0.01 wt % to 0.20 wt % of the 9-des-methyl impurity; and
   from 0.01 wt % to 0.20 wt % of the 10-des-methyl impurity,
wherein the wt % is relative to the total weight of the particles.
39. A compound for use according to any one of the preceding embodiments, wherein the compound is used in combination with a muscarinic receptor antagonist, a beta-adrenergic receptor agonist or an inhaled corticosteroid.
40. A method of treating chronic obstructive pulmonary disease (COPD) in a patient, the method comprising administering a therapeutically effective amount of a compound to the patient, which compound is ensifentrine or a pharmaceutically acceptable salt thereof, wherein the patient has a blood eosinophil count of <300 cells/μL.
41. Use of a compound in the manufacture of a medicament for use in a method of treating chronic obstructive pulmonary disease (COPD) in a patient, which compound is ensifentrine or a pharmaceutically acceptable salt thereof, wherein the patient has a blood eosinophil count of <300 cells/μL.
42. A kit comprising a pharmaceutical composition comprising ensifentrine or a pharmaceutically acceptable salt thereof and instructions for use in treating COPD in a patient having a blood eosinophil count of <300 cells/μL.

We claim:

1. A method of treating chronic obstructive pulmonary disease (COPD) in a subject, the method comprising administering to the subject a composition comprising ensifentrine or a pharmaceutically acceptable salt thereof,
wherein the subject has a blood eosinophil count of less than 300 cells/μL,
wherein the method reduces frequency of COPD exacerbations or severity of COPD exacerbations in the subject compared to frequency of COPD exacerbations or severity of COPD exacerbations in an untreated subject having COPD and not administered the composition.

2. The method of claim 1, wherein the method decreases the frequency of COPD exacerbations or the severity of COPD exacerbations in the subject by at least 20% compared to the frequency of COPD exacerbations or the severity of COPD exacerbations in the untreated subject having COPD and not administered the composition.

3. The method of claim 2, wherein the method decreases the frequency of COPD exacerbations or the severity of COPD exacerbations in the subject by at least 40% compared to the frequency of COPD exacerbations or the severity of COPD exacerbations in the untreated subject having COPD and not administered the composition.

4. The method of claim 1, wherein the method increases time to first COPD exacerbation in the subject compared to time to first COPD exacerbation in the untreated subject having COPD and not administered the composition.

5. The method of claim 1, wherein the subject has a blood eosinophil count of less than 150 cells/μL.

6. The method of claim 1, wherein the subject has a blood eosinophil count of less than 100 cells/μL.

7. The method of claim 1, wherein the blood eosinophil count is determined from a sample of blood obtained from the subject using a Coulter counter.

8. The method of claim 1, wherein the subject is susceptible to COPD exacerbations.

9. The method of claim 1, wherein the subject has suffered one or more COPD exacerbations in a year preceding the administering to the subject.

10. The method of claim 1, wherein the subject is susceptible to pneumonia.

11. The method of claim 1, wherein the subject is not on an inhaled corticosteroid regimen at a time of the administering to the subject.

12. The method of claim 1, wherein the composition is administered to the subject via inhalation.

13. The method of claim 1, wherein the administering to the subject is once, twice or three times per day.

14. The method of claim 13, wherein the administering to the subject is twice a day.

15. The method of claim 1, wherein the administering to the subject comprises a total daily dose of the ensifentrine or the pharmaceutically acceptable salt thereof of from 0.5 to 10 mg.

16. The method of claim 15, wherein the administering to the subject comprises a dose of about 3 mg of the ensifentrine or the pharmaceutically acceptable salt thereof to the subject twice a day.

17. The method of claim 1, wherein the administering to the subject comprises administering the ensifentrine or the pharmaceutically acceptable salt thereof to the subject at least once per day for at least 8 weeks, at least 16 weeks, or for at least 24 weeks.

18. The method of claim 1, wherein the composition is an inhalable pharmaceutical composition comprising a diluent.

19. The method of claim 18, wherein the inhalable pharmaceutical composition comprises:
(a) particles comprising the ensifentrine or the pharmaceutically acceptable salt thereof at a concentration of 1.2 mg/mL;
(b) polysorbate 20 at a concentration of 0.5 mg/mL;
(c) sorbitan monolaurate at a concentration of 0.05 mg/mL;
(d) sodium dihydrogen phosphate dihydrate at a concentration of 0.744 mg/mL;
(e) disodium hydrogen phosphate dihydrate at a concentration of 0.853 mg/mL; and
(f) sodium chloride at a concentration of 8.6 mg/mL.

20. The method of claim 1, wherein the composition is administered to the subject in combination with a muscarinic receptor antagonist, a beta-adrenergic receptor agonist or an inhaled corticosteroid.
